(19) United States
(12) Patent Application Publication
(10) Pub. No.: US 2005/0003461 A1
(43) Pub. Date: Jan. 6, 2005

(54) Method for detecting allergies

(76) Inventor: Hans-Joerg Buehring, Tuebingen (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/861,892

(22) Filed: Jun. 4, 2004

Related U.S. Application Data
(63) Continuation of application No. PCT/EP02/13437, filed on Nov. 28, 2002.

(30) Foreign Application Priority Data
Dec. 6, 2001 (DE) 101 60 921.3

(51) Int. Cl. G01N 33/567

(52) U.S. Cl. 435/7.21

(57) ABSTRACT

The present invention relates to a method for detecting allergies, with a blood sample initially being incubated with a cell-influencing agent, preferably interleukin IL-3, and then being incubated with an allergen, and with this blood sample subsequently being incubated with an antibody which binds to the surface structures on basophils and/or mast cells and/or precursor cells of basophils and/or mast cells to which the antibody 97A6 can bind, which antibody is produced and released by hybridoma cells which were deposited in the Deutsche Sammlung von Mikroorganismen und Zellkulturen [German collection of microorganisms and cell cultures] GmbH, DSMZ, under number DSM ACC 2297, in accordance with the Budapest Treaty, on Dec. 2, 1997, and with the antibodies which have bound to the basophils and/or mast cells and/or precursor cells of the basophils and/or mast cells then being quantified.

### CROSS REFERENCES TO RELATED APPLICATIONS

This application is a continuation of copending International Patent Application PCT/EP 02/13437 filed on Nov. 28, 2002, and designating the U.S., which was not published under PCT Article 21(2) in English, and claims priority of German patent application DE 101 60 921.3 filed on Dec. 6, 2001, which is incorporated herein by reference.

### FIELD OF THE INVENTION

The invention relates to a method for detecting allergies.

### DESCRIPTION OF THE RELATED ART

WO 00/72010 discloses a method of this nature.

Nowadays, allergies affect well over 20% of the world's population, and in the western countries, in particular, there are increasingly more allergic diseases both in childhood and in adulthood.

Allergic reactions can be induced, for example, by pollen, animal hair and scales, nutrients and drugs, house dust mites, insect poisons, etc. The allergic reactions which are induced manifest themselves, for example, as a rhinitis or a skin rash, or even lead to an allergic shock usually depending on the body regions with which the allergens come into contact.

In allergic patients, wasp and bee stings, in particular, can elicit intense local swellings or severe systemic disturbances which encompass the entire organism. In extreme cases, allergic reactions can even lead to death.

Allergic reactions occur when an individual, who has already produced IgE antibodies as a response to a harmless antigen, subsequently comes into contact with the same allergen once again. This second exposure initiates a stage in the hypersensitivity reaction.

The immediate-type allergies are due to hypersensitivity reactions which are mediated by IgE. The IgE molecules which are formed, after contact with an allergen, in an overshooting reaction bind, in particular, to the corresponding IgE receptors (“high-affinity receptors”, Fc,RI) on mast cells and on basophilic granulocytes (basophils). When a second contact is made with the allergen, the latter binds the IgE molecules which are bound to the cell receptors and crosslinks the IgE molecules, thereby inducing activation of these cells. The same reaction can also be induced deliberately using anti-IgE antibody instead of an allergen.

This results in the activated cells secreting preformed chemical mediators, such as histamine, which play an active role in the pathogenesis of hypersensitivity reactions.

The extent to which basophils release histamine varies between patients. The basophils of some patients do not in fact release any histamine at all in response to anti-IgE; these patients are termed “nonresponders”. The precise steps which are responsible for this lack of release have not yet been clarified. However, it has been shown that the density of IgE on the basophil surfaces in nonresponders is about the same as the density of IgE in responders, which means that the lack of release cannot be attributed, for example, to a lower number of IgE receptors.

Yamaguchi M. et al. 1996 “Nonreleasing Basophils Convert to Releasing Basophils by Culturing with IL-3”, J. Allergy Clin. Immunol. 97:1279-1287, reported that histamine release could also be induced in the case of nonresponder basophils if the latter were stimulated with interleukin IL-3.

The diagnosis of Hymenoptera allergies, that is allergies to venoms produced by membrane-winged insects, such as bees or wasps, is chiefly based on recording prior history, carrying out skin tests and determining specific IgE antibodies. In selected cases, the allergen-induced release of histamine or leukotriene C4 is measured in order to confirm an allergic status.

Other tests are based on flow-cytometric analyses, in order either to determine the binding of fluorescence-labelled allergens to specific, cell-bound IgE molecules or to determine an allergen-induced cell-surface expression of basophilic granule antigens.

Thus, Pâris-Köhler A. et al. 2000 “In vitro Diagnosis of Cypress Pollen Allergy by using Cytofluorimetric Analysis of Basophils (Basotest)”, J. Allergy Clin. Immunol. 105:339-345, and Knol E. et al. 1991 “Monitoring Human Basophil Activation via CD63 Monoclonal Antibody 435”, J. Allergy Clin. Immunol. 88:328-338, for example, describe the induced cell-surface expression of the granule-associated molecule CD63 on the surface of activated basophils.

Irsch J. et al. 1999 “The Frequency of Phospholipase A2 Binding of Basophilic Granulocytes does not decrease during Bee-venom-specific Immunotherapy”, Allergy 54:742-747, showed that phospholipase A2 bound directly to the basophils of patients who were allergic to bee venom.

The abovementioned tests are suitable for analyzing the binding of molecularly de-fined allergens in order to detect immediate-type hypersensitivity and also possibly to investigate the success of a hyposensitization in allergic patients. In the case of a hyposensitization, the patient is injected with increasing doses of an allergen to which he/she reacts oversensitively.

WO 00/72010, which was mentioned at the outset, describes the use of a monoclonal antibody for detecting allergy. In this connection, use is made of the fact that, when basophils are activated by an allergen, particular surface structures on the cells are ex-pressed more strongly. The antibody which is described in this document binds to these more strongly expressed surface structures and can consequently also be used to quantify activated and nonactivated basophils. The document also describes a method for detecting allergies which uses this antibody.

A disadvantage of this method, and of the abovementioned tests, is that they cannot be used for nonresponders. As explained above, no release of histamine, and no upregulation of surface structures, takes place in nonresponders even though the patients concerned can be allergic to a particular allergen. While nonresponders can be positive in skin tests or produce specific IgE antibodies, they cannot be identified as being allergic using a method which detects activated and nonactivated basophils or the upregulation of particular surface structures.

Thus, after a test which uses the customary methods, there always remains a degree of uncertainty as to whether the patients who are identified from their samples as being nonresponders are also in fact patients who are not allergic.

### SUMMARY OF THE INVENTION

In view of the above, an object of the present invention is to provide a method which can be used to successfully diagnose the nonresponders among allergic patients as well.

According to the invention, this object is achieved by means of a method for detecting allergies, which comprises the steps of:
incubating a blood sample with an agent that has an effect on cells, preferably with interleukin IL-3,
incubating this blood sample with an allergen,
incubating this blood sample with an antibody which binds to the surface structures on basophils and/or mast cells and/or on precursor cells of basophils and/or mast cells to which the antibody which is designated 97A6 can bind, which antibody is produced and released by hybridoma cells which were deposited in the Deutsche Sammlung von Mikroorganismen und Zellkulturen [German collection of microorganisms and cell cultures] GmbH, DSMZ, under number DSM ACC 2297, in accordance with the Budapest Treaty, on Dec. 2, 1997,
quantifying the antibodies which are bound to the basophils and/or mast cells and/or precursor cells of the basophils and/or mast cells.

### DETAILED DESCRIPTION OF THE PREFERRED EMBODIMENT

The object underlying the invention is completely achieved in this way.

Thus, the inventors of the present application have found it possible, following a treatment of nonresponders with interleukin IL-3 and subsequent incubation with a given allergen, to upregulate the surface structure on basophils and/or mast cells and/or precursor cells of basophils and/or mast cells to which antibody 97A6 can bind. While interleukin IL 3 itself induces upregulation of this surface structure, the additional upregulation achieved by the allergen is greater by at least a factor of two and can therefore be detected using measuring equipment.

The conventional tests, i.e. tests in which the status of basophils is determined, are unable to detect the allergic patients among the nonresponders since, in the case of these patients, no histamine is released following contact with allergen and nor is a given surface structure upregulated, in order to confirm an allergic status.

The inventors demonstrated that it was possible to use the method according to the invention to upregulate a given surface structure in the nonresponders who were allergic. It was therefore possible, by subsequently incubating with an antibody which recognizes the upregulated surface structure, to identify the allergic patients among the nonresponders and to classify the type of allergy unambiguously.

Following incubation with interleukin IL 3, the surface structure in nonallergic nonresponders was not specifically upregulated by an allergen, which meant that it was also possible to confirm these nonresponders as in fact being nonallergic patients.

Interleukins are cytokines which are mainly secreted by leukocytes following physiological or nonphysiological stimuli and affect the proliferation, differentiation and cell-cell interactions of lymphocytes and their precursor cells from the bone marrow and also of other blood cells.

While interleukin IL 3 is mainly formed by antigen-activated T lymphocytes, it is also formed, for example, by endothelial cells, mast cells, monocytes, etc. It has a powerful influence on the growth and differentiation of hematopoietic stem cells and promotes the release of histamine and leukotriene C4 from basophilic granulocytes.

By carrying out measurements in comparison with interleukin EL 3, it is possible to identify other agents which have the effect according to the invention. These agents include those which influence cells by means, for example, of a proliferating and/or stimulating and/or activating effect.

The inventors were able to show that incubating blood samples with interleukin IL-3 upregulated surface structures to which antibody 97A6 binds. The surface structure is phosphodiesterase/nucleotide pyrophosphatase-3 (PDNP3), which is also designated E NPP3 or PD-Tbeta. The sequence was published by Jin-Hua et al. in Genomics 45:412-415 (1997).

It is another object of the invention to use a quantity of interleukin IL 3 of from 1 to 20 ng/ml, preferably of approx. 10 ng/ml within the method according to the invention.

Still a further object of the invention relates to the incubation of the blood sample with interleukin IL 3 for between one hour and 96 hours, preferably overnight, that is for approx. 24 hours.

The inventors were able to show that, at 24 h, the stimulation index was optimal for carrying out the tests in the case of responders as well.

It is a further object of the invention to use, as antibody, a monoclonal antibody, in particular the antibody 97A6, which is produced and released by hybridoma cells which were deposited in the Deutsche Sammlung von Mikroorganismen und Zellkulturen [German collection of microorganisms and cell cultures] GmbH, DSMZ, under number DSM ACC 2297, in accordance with the Budapest Treaty, on Dec. 2, 1997. The storage period was extended correspondingly.

This antibody has already been tested in clinical practice and is marketed by the company Beckman Coulter under order number IM3575.

This antibody specifically recognizes an epitope of the surface structure phosphodiesterase/nucleotidepyrophosphatase-3 (PDNP3), which is upregulated in an allergen-specific manner in allergic patients.

It is another object of the invention to use an antibody which binds to an epitope of PDNP3 which is different from that to which antibody 97A6 binds.

Advantageously, an amplification of the detection signal can be achieved thereby since more antibodies can then bind to a cell.

It is a further object of the invention to use an antibody which is linked to a label, in particular to a fluorescent label.

As a result, the antigen can be detected with a high degree of sensitivity, for which reason only small quantities of the antibody have to be used for the detection.

Bound antibodies are then detected, for example, by means of customary immunological detection methods, for example ELISA (enzyme-linked immunosorbent assay) or by means of a FACS analysis. Using these detection methods offers the advantage of analyzing cells in a highly specific, rapid and sensitive manner.

Still a further object of the invention relates to the use of the method for detecting allergies in the case of samples which were identified in allergy tests as being nonresponders.

Using this method after a first allergy test, without any incubation with interleukin IL 3, has the advantage that, in an economical manner, the method can be specifically used only for the samples which have been found to be nonresponders in the first test. Carrying out the method according to the invention after this first test ensures that it is also possible to identify the nonresponders among the allergic patients.

It is a further object of the invention to use IL-3 for detecting allergies in samples which—in a previous test with the allergen—displayed a nonresponder status.

Another object of the invention is to use an antibody which binds to the surface structures on basophils and/or mast cells and/or precursor cells of basophils and/or mast cells to which the antibody designated 97A6 is able to bind for detecting allergies of nonresponders.

It will be understood that the abovementioned features and those which are still to be explained below can be used not only in the combinations indicated but also in other combinations, or alone, without leaving the scope of the present invention.

Further advantages are evident from the following embodiment.

### EXAMPLE

Blood cells from nonresponders (allergic patients and normal individuals) and from responders were stimulated for different periods of time (one hour, 24 hours, 48 hours and 96 hours) with 10 ng/ml of interleukin IL 3 at a temperature of 37° C. (3 ml of whole peripheral blood in 10 ml of whole medium plus 10 ng/ml of interleukin IL 3).

The incubation was carried out in 25 cm² tissue culture flasks at 37° C. and at 5% CO₂. The expression of E NPP3 was measured at the time points of zero hours (prior to incubation), one hour, one day, two days, three days (or four days) and six days after incubation with interleukin IL 3.

Subsequently, the samples were in each case incubated, at 37° C. for 15 minutes, with serial dilutions of bee and wasp venom or PBS (phosphate-buffered saline) plus calcium (negative control) or anti-IgE (positive control).

The reaction was stopped with 20 mM EDTA solution and the cells were resuspended in 50 μl of FACS buffer (0.1% NaN₃+0.1% BSA in Hank's balanced salt solution). The cells were then incubated, at room temperature for 15 min, with 10 μl of 97A6-PE antibody (final concentration, 1 μg/ml).

2 ml of erythrocyte lysis reagent (Versalyse, Immunotech) were then added and the sample was incubated at room temperature for 10 min. After that, 2 ml of FACS buffer were added and the sample was centrifuged at 1200 rpm for 7 min. The supernatant was discarded and 4 ml of FACS buffer were added to the pellet; the sample was then centrifuged once again at 1200 rpm for 7 min. After the supernatant had been discarded, the cell-containing pellet was taken up in 150 μl of FACS buffer and stored on ice.

After that, the cells were analyzed by flow cytometry (FACScalibur). The change in the expression of E-NPP3 was determined while calculating the stimulation index (SI) and the percentage of stimulated basophils. The stimulation index is calculated from: mean fluorescence intensity of allergen-stimulated or anti-IgE-stimulated 97A6⁺ cells/mean fluorescent intensity of PBS-stimulated 97A6⁺ cells.

Result:

The results showed that the optimal stimulation period for determining the allergen-specific upregulation of E NPP3 was 24 hours (optimal stimulation index).

For the tested allergic and nonallergic nonresponders, it was not possible to observe any significant improvement in the anti-IgE-induced upregulation of E-NPP3 even after IL-3 stimulation.

However, in the available allergic nonresponders, it was surprisingly possible to observe an allergen-specific upregulation of E NPP3 after stimulation with IL 3. In both cases, it was possible to confirm the allergy type (in each case, wasp and not bee) which, according to the anamnesis, was ascertained in a skin test and by determining specific IgE.

1. A method for detecting allergies, which comprises the steps of:
incubating a blood sample with an agent that has an effect on cells,
incubating this blood sample with an allergen,
incubating this blood sample with an antibody which binds to the surface structures on basophils and/or mast cells and/or on precursor cells of basophils and/or mast cells to which the antibody 97A6 can bind, which is produced and released by hybridoma cells which were deposited in the Deutsche Sammlung von Mikroorganismen und Zellkulturen GmbH, DSMZ, under number DSM ACC 2297, and
quantifying the antibodies which are bound to the basophils and/or mast cells and/or precursor cells of the basophils and/or mast cells.

2. The method of claim 1, wherein said agent that has an effect on cells is interleukin IL-3.

3. The method of claim 2, wherein a quantity of interleukin IL-3 is from 1 to 20 ng/ml is used.

4. The method of claim 3, wherein the quantity of interleukin IL-3 is about 10 ng/ml.

5. The method of claim 2, wherein the blood sample is incubated with interleukin IL 3 for between one hour and 96 hours.

6. The method of claim 5, wherein the blood sample is incubated with interleukin IL 3 for about 24 hours.

7. The method of claim 1, wherein the antibody employed is a monoclonal antibody.

8. The method of claim 1, wherein the antibody employed is the antibody 97A6, which is produced and released by hybridoma cells which were deposited in the Deutsche Sammlung von Mikroorganismen und Zellkulturen GmbH, DSMZ, under number DSM ACC 2297.

9. The method of claim 1, wherein the antibody employed is an antibody which binds to epitopes of the surface structures which are different from that to which the antibody 97A6 binds.

10. The method of claim 1, wherein the antibody is linked to a label.

11. The method of claim 10, wherein said label is a fluorescent label.

12. The method of claim 1, wherein said blood sample is from patient identified in allergy tests as being a nonresponder.

13. A method for detecting allergies in samples which have been identified in allergy tests as being nonresponders, comprising the step of contacting the samples with an antibody which binds to the surface structures on basophils and/or mast cells and/or precursor cells of basophils and/or mast cells to which the antibody designated 97A6 can bind.

14. The method of claim 13, further comprising
incubating a blood sample with interleukin IL-3,
incubating this blood sample with an allergen,
incubating this blood sample with an antibody which binds to the surface structures on basophils and/or mast cells and/or on precursor cells of basophils and/or mast cells to which the antibody 97A6 can bind, said antibody produced and released by hybridoma cells which were deposited in the Deutsche Sammlung von Mikroorganismen und Zellkulturen GmbH, DSMZ, under number DSM ACC 2297, and
quantifying the antibodies which are bound to the basophils and/or mast cells and/or precursor cells of the basophils and/or mast cells.

15. A method for detecting allergies in samples which have been identified in allergy tests as being nonresponders, comprising the step of contacting the samples with interleukin IL 3.
